(19) United States
(12) Patent Application Publication
(10) Pub. No.: US 2008/0234570 A1
(43) Pub. Date: Sep. 25, 2008

(54) System For Guiding a Medical Instrument in a Patient Body

(75) Inventors: Olivier Gerard, Viroflay (FR); Raoul Florent, Ville D'Avray (FR)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS, N.V., EINDHOVEN (NL)

(21) Appl. No.: 10/598,327

(22) PCT Filed: Jan. 24, 2005
(86) PCT No.: PCT/IB2005/000498

§ 371 Date: Aug. 24, 2006

(30) Foreign Application Priority Data
Mar. 5, 2004 (EP) 04300119.7

(51) Int. Cl. A61B 6/12

(52) U.S. Cl. 600/424

(57) ABSTRACT

The present invention relates to a medical system comprising a medical instrument to be guided in a patient body, ultrasound acquisition means using an ultrasound probe for acquiring a 3D ultrasound data set and X-ray acquisition means for acquiring a 2D X-Ray image, which comprises a projection of said medical instrument. The system in accordance with the invention further comprises means for localizing said ultrasound probe within a referential of said X-Ray acquisition means, means for providing a first localization of said medical instrument within a referential of the ultrasound acquisition means, means for converting said first ultrasound localization into a first X-Ray localization within the referential of said X-Ray acquisition means, means for providing a second X-Ray localization of the projection of said medical instrument in the two-dimensional X-ray image, means for mapping said 3D ultrasound data set with said 2D X-ray image in accordance with a transformation, which minimizes a distance between a projection of said first X-Ray localization on said 2D X-Ray image and said second X-Ray localization and means for generating and displaying a bi-modal representation of said medical instrument, in which said 2D X-ray image and said mapped 3D ultrasound data are combined.

### FIELD OF THE INVENTION

The present invention relates to a medical system. The invention also relates to a method to be used in said system. The invention finds for example its application for guiding a catheter inside the heart of a patient during an electrophysiology interventional procedure.

### BACKGROUND OF THE INVENTION

Clinical applications in which a medical instrument has to be guided into the body of a patient are becoming widespread. Notably the growing interest in minimal-invasive methods for the treatment of cardiac diseases necessitates the development of methods and devices allowing the physician to guide a medical instrument to predetermined positions inside or outside the heart. In electrophysiology for example, it is necessary to guide a catheter to a plurality of predetermined positions on the ventricular or atrial walls in order to measure an electrical pulse or to burn wall tissues.

U.S. Pat. No. 6,587,709 discloses a system for guiding a medical instrument in the body of a patient. Such a system acquires a live 3D ultrasound image data set using an ultrasound probe. An advantage of acquiring a 3D image data set is to get depth information. An advantage of using a live 3D ultrasound image modality is that the surrounding anatomy is visible, which facilitates the guidance of the medical instrument by the physician. The system further comprises localization means for localizing the medical instrument within the 3D ultrasound data set, which locates three ultrasound receivers mounted on the medical instrument relatively to said ultrasound probe. Such a localization allows an automatic selection of a plane to be imaged, which comprises at least a section of the medical instrument. Therefore no readjustment of the ultrasound probe position by hand is necessary.

A first drawback of such a 3D ultrasound data set is to have a narrow viewing field, which does not cover the whole part of the patient body concerned by a catheter introduction and placement. Therefore, for guiding the catheter during the whole procedure, the ultrasound probe has to be moved several times. At each displacement, a pre-operative step of locating the ultrasound probe in a referential of the interventional room is needed, because the location of the catheter is measured relatively to the ultrasound probe location. Such a pre-operative step may delay and complicate the interventional procedure.

A second drawback of the ultrasound imaging modality is to have a low resolution Therefore, the acquired 3D ultrasound data set does not give an image of the catheter and its surrounding of acceptable quality.

A third drawback of the ultrasound imaging modality is that there are some zones of the patient body where the thoracic cage blocks the ultrasound scan and no exploitable image can be output.

### SUMMARY OF THE INVENTION

The object of the invention is therefore to provide a system for guiding a medical instrument in a patient body, which gives an improved visibility of the medical instrument and its surrounding anatomy during the whole procedure.

This is achieved by a medical system comprising:
a medical instrument to be guided in a patient body,
X-Ray acquisition means for acquiring a two-dimensional X-ray image comprising a projection of said medical instrument in accordance with a geometry of said X-Ray acquisition means,
ultrasound acquisition means for acquiring a three-dimensional ultrasound data set of said medical instrument using an ultrasound probe,
means for localizing said ultrasound probe within a referential of the X-ray acquisition means,
means for providing a first ultrasound localization of said medical instrument within a referential of said ultrasound acquisition means,
means for converting said first ultrasound localization within said referential of the ultrasound acquisition means into a first X-ray localization within said referential of the X-ray acquisition means, using said localization of the ultrasound probe,
means for providing a second X-ray localization of said projection of the medical instrument in a referential of said two-dimensional X-ray image,
means for mapping said three-dimensional ultrasound data set with said two-dimensional X-ray image in accordance with a transformation, which minimizes a distance between a projection of said first X-ray localization on said two-dimensional X-Ray image in accordance with said geometry of the X-Ray acquisition means and said second X-ray localization,
means for generating and displaying a bi-modal representation of said medical instrument in which said two-dimensional X-ray image and said mapped three-dimensional ultrasound data set are combined.

With the invention, a bimodal representation is provided, in which two-dimensional (2D) X-Ray data and three-dimensional (3D) ultrasound data are combined. 2D X-ray data provide a good visibility and a high resolution of the medical instrument and of bone structures. 2D X-Ray data also benefit from a large viewing field, which allows a visualization of the whole area of the patient body concerned by the electrophysiology procedure.

3D ultrasound data provide a good visibility of soft tissues and vascularities in a surrounding of the medical instrument. In addition, 3D ultrasound data give an indication of depth, which is not provided by the 2D X-Ray image, because said X-Ray image only provides a projection of said medical instrument in accordance with a geometry of the X-Ray acquisition means. Such a geometry defines lines of projection, along which absorptions of X-rays by the exposed tissues of the patient are accumulated.

Therefore, the visibility of the surrounding of the medical instrument is improved by the combination of the 2D X-Ray and the 3D ultrasound data.

In order to provide such a combination, the system firstly localizes the ultrasound probe and the 3D ultrasound data set in a referential of the X-Ray acquisition means. Such a referential of the X-ray acquisition means is supposed to be fixed. Therefore, assuming that the ultrasound probe does not move, a position of any point of the 3D ultrasound data set is known in said referential the X-ray acquisition means.

The system in accordance with the invention further provides a first ultrasound localization of the medical instrument in the 3D ultrasound data set. Such a first ultrasound localization is expressed with coordinates of a referential of the 3D ultrasound acquisition means. The first ultrasound localization is then converted into a first X-Ray localization of the medical instrument within the referential of the X-Ray acquisition system, using the localization of the ultrasound probe.

The system in accordance with the invention also provides a second X-Ray localization of the projection of the medical instrument in the 2D X-ray image, which is expressed with coordinates of a referential of the 2D X-Ray image, for instance a referential of the detector. Such a referential is known by the geometry of the X-Ray acquistion means. Therefore, the geometry allows to determine a projection of any point of the referential of the X-Ray acquisition means and, inversely, a point of the detector corresponds to a line of projection within the referential of the X-Ray acquisition means.

From said first X-Ray and second X-Ray localizations the mapping means are intended to define a transformation, which minimizes a distance between a projection of said first X-ray localization on the two-dimensional X-Ray image in accordance with the geometry of the X-Ray acquisition means and said second X-ray localization. Such a transformation is applied to the 3D ultrasound data set. Finally the system generates and display a bimodal representation in which the 2D X-ray image and the transformed 3D ultrasound data set are combined by affecting to a point of the bimodal representation either ultrasound data or X-ray data or a combination of both.

An advantage of such a transformation is to compensate for errors in the localization of the ultrasound probe. These errors may be due either to any external or internal movement, like respiratory movements, which could have occurred after the localization of the ultrasound probe in the referential of the X-Ray acquisition system, or to an imprecision in the localization of the ultrasound probe, for instance related to its orientation. Consequently the mapping of 3D ultrasound and 2D X-ray data in the surrounding of the medical instrument is made more precise. In particular, the distance between the medical instrument and the wall tissues shown by the bimodal representation becomes more accurate and reliable, which is of high interest for guiding the medical instrument into contact with a wall tissue.

In a first embodiment of the invention, the localization of the medical instrument in both 3D ultrasound data set and 2D X-Ray image is based on a detection of one landmark, for instance a tip usually placed at one extremity of the medical instrument. Such a localization allows to define a translation for mapping the 3D ultrasound data set with the 2D X-ray image. An advantage of this first embodiment is that it is very simple and easy to implement.

In an alternative, the system in accordance with the invention further comprises means for detecting an orientation of the medical instrument, which is defined by two Euler angles. Therefore, a transformation can be specified, which comprises a translation and two rotations.

In a second embodiment of the invention, the first and second localizations of the medical instrument are based on a plurality of landmarks, which are for instance arranged at different places on the medical instrument. An advantage is that a transformation comprising a translation and 3 rotations can be defined, which is enough to completely specify a displacement of the 3D ultrasound data set in the referential of the X-Ray acquisition means. Therefore, in a surrounding of the medical instrument, the mapping of ultrasound and X-ray data is improved.

In a third embodiment of the invention, the plurality of landmarks are placed on the medical instrument and on at least two reference medical instruments. An advantage is that the two reference medical instruments are expected to be fixed. Consequently, any displacement of a landmark of a reference medical instrument with respect to the anatomy may advantageously be considered as an indication that the ultrasound probe has moved and more generally that the mapping of the 3D ultrasound data set with the 2D X-ray image is no more reliable. Another advantage is that the landmarks used for localizing the medical instrument are more distant from each other. Therefore, the definition of the transformation is more robust to local errors of localization. Therefore, a mapping transformation can be defined, which applies to a larger surrounding of the medical instrument and the precision of the bimodal representation is improved on a larger area.

These and other aspects of the invention will be apparent from and will be elucidated with reference to the embodiments described hereinafter.

### BRIEF DESCRIPTION OF THE DRAWINGS

The present invention will now be described in more detail, by way of example, with reference to the accompanying drawings, wherein:

FIG. 1 is a schematical drawing of a system in accordance with the invention,

FIG. 2 is a schematical drawing of means for localizing the ultrasound probe within the X-ray referential, when the ultrasound probe is equipped with active localizers,

FIGS. 3, 4a and 4b are schematical drawings of means for localizing the ultrasound probe and the 3D ultrasound data set within the referential of the X-Ray acquisition means, when the ultrasound probe is equipped with a belt comprising radio-opaque markers,

FIG. 5 is a schematical drawing of means for providing a first localization of the medical instrument within the 3D ultrasound data set,

FIG. 6 is a schematical drawing of means for providing a second localization of the projection of the medical instrument in a referential of the 2D X-ray image,

FIG. 7 is a schematical drawing of mapping means for mapping the 3D ultrasound data set with the 2D X-ray image when the transformation is a translation,

FIG. 8 is a schematical drawing of means for providing a first localization of the medical instrument within the 3D ultrasound data set, when a plurality of landmarks are placed on the medical instrument and two reference instruments,

FIG. 9 is a schematical drawing of means for generating a bimodal representation in accordance with the invention,

FIG. 10 is a schematical drawing of means for generating a bimodal representation, when the system in accordance with the invention comprises means for segmenting a wall tissue region around the medical instrument,

FIG. 11 is a functional diagram of the method in accordance with the invention.

### DETAILED DESCRIPTION OF THE INVENTION

The present invention relates to a medical system comprising a medical instrument to be guided in a patient body and data acquisition and processing means for visualizing said medical instrument. Such a system is particularly adapted for guiding a catheter within the heart cavities in order to diagnose and cure heart diseases, but it can more generally be used for guiding any other medical instrument in the patient body, like for instance a needle.

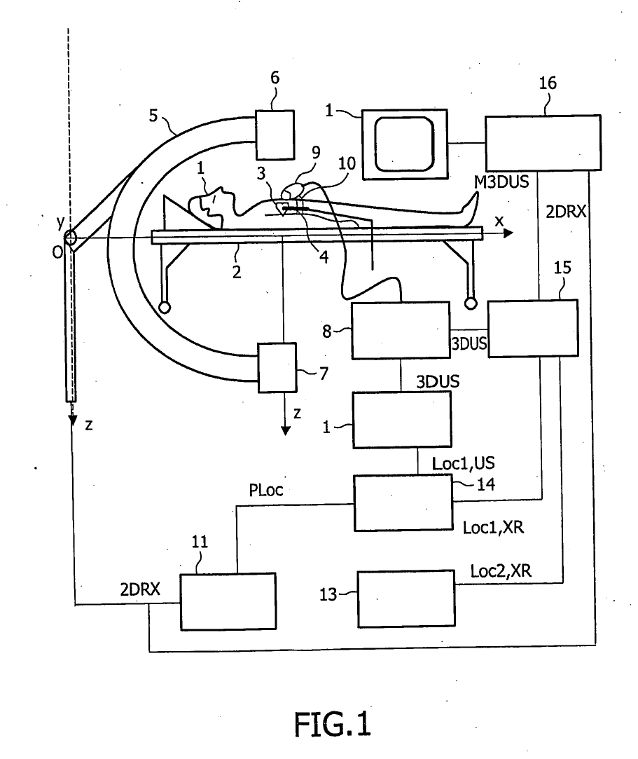

The schematical drawing of FIG. 1 shows a patient 1, who is arranged on a patient table 2 and whose symbolically indicated heart 3 is subjected to a treatment by means of a catheter 4 introduced into the body. The system comprises means 5 for acquiring a 2D X-ray image of the patient body. Said X-ray acquisition means comprise a focal X-ray source 6 and a detector 7. Advantageously, these X-ray acquisition means 5 comprise a C-arm system, as it is usually the case in a cathlab room. An advantage of such a C-arm system is to be able to have a rotational motion around the patient body in order to produce a plurality of 2D X-ray images of the patient at known orientation angles.

The system in accordance with the invention further comprises means 8 for acquiring a 3D ultrasound data set from an ultrasound probe 9, which has been placed on the patient body and fixed by fixation means, for instance a belt 10 or a stereotactic arm. It should be noted that both 2D X-ray image and 3D ultrasound data set are acquired in real-time, which enables a live visualization of the medical instrument, when it is guided inside the patient body.

The X-ray acquisition means 5 comprise a referential of coordinates (O, x, y, z), called X-Ray referential hereinafter, in which the geometry of the focal X-ray source 6 and the detector 7 is known. It should be noted that the X-Ray referential (O, x, y, z) is bound to the fixed part of the X-Ray acquisition means and not to the C-arm. Therefore, the orientation of the C-arm can be expressed in said X-Ray referential. However, the geometry of the X-Ray acquisition means is dependent on a particular position of the C-arm.

The system in accordance with the invention further comprises means 11 for localizing the ultrasound probe 9 within the X-Ray referential (O, x, y, z), means 12 for providing a first ultrasound localization Loc_{1,US} of the catheter 4 in the 3D ultrasound data set within a referential of the ultrasound acquisition means, means 13 for providing a second X-Ray localization Loc_{2, XR} of a projection of the catheter 4 in the 2D X-ray image within a referential of the 2D X-Ray image or of the detector, means 14 for converting said first ultrasound localization Loc_{1,US} into a first X-Ray localization of the medical instrument 4 within the X-Ray referential, means 15 for mapping said 3D ultrasound data set with said 2D X-ray image in accordance with a transformation, which minimizes a distance between a projection of said first X-Ray localization on the 2D X-Ray image in accordance with the geometry of the X-Ray acquisition means and said second X-Ray localization. The system in accordance with the invention finally comprises means 16 for generating and displaying a bi-modal representation BI of the catheter 4 in which the 2D X-ray image and the mapped 3D ultrasound data are combined. The bimodal image BI is displayed on a screen 17.

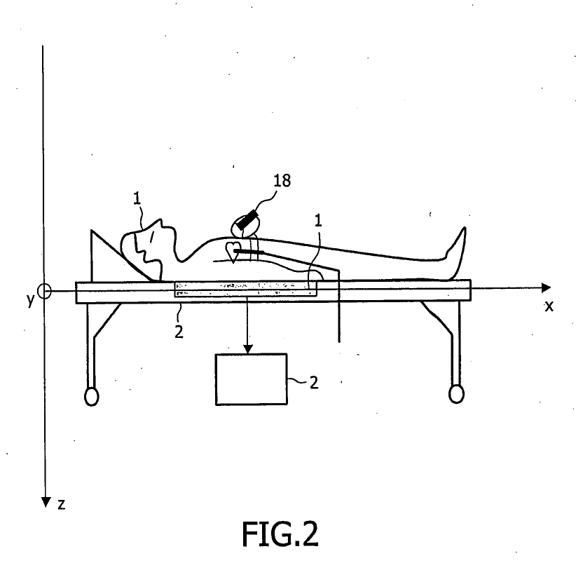

Referring to FIG. 2, the probe localization means 11 are, in a first approach, based on an active localizer 15, well-known to those skilled in the art, which is arranged on the ultrasound probe 9. Said active localizer 18, for instance an RF coil, is intended to transmit an RF signal to an RF receiving unit 19 placed under the patient body and for instance integrated into the table. The RF receiving unit transmits the received signal to measuring means 20 for measuring a position of the ultrasound probe 9 in a known referential, for instance the X-Ray referential (O, x, y, z). It should be noted that the active localizer 18 must be two-dimensional and placed on the ultrasound probe 9 in such a way that a precise measurement of the position and of the orientation of the ultrasound probe can be calculated. An advantage of this first approach is to provide a precise localization of the ultrasound probe 9.

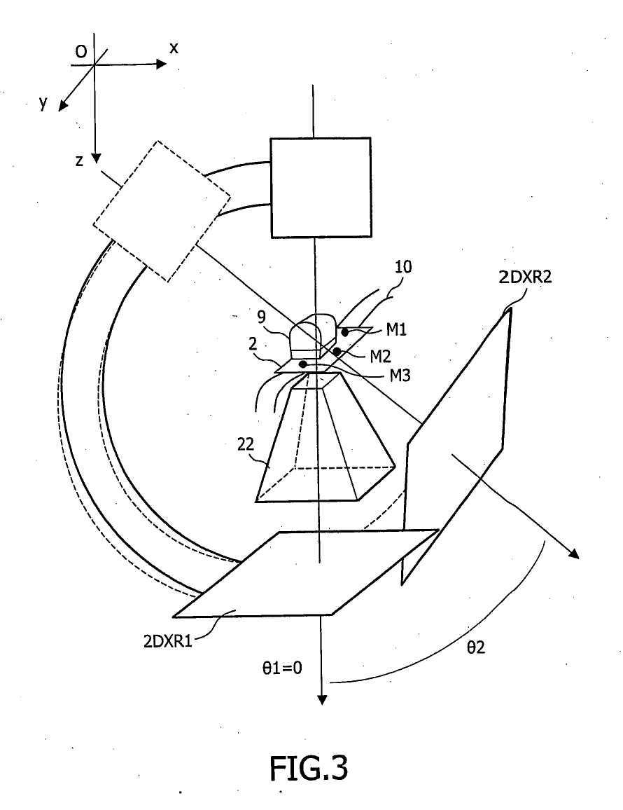

In a second approach of the probe localization means 11 shown in FIG. 3, the ultrasound probe 9 is fixed around the body of the patient 1 with a belt 10 equipped with at least three non aligned interdependent radio-opaque markers M₁, M₂ and M₃. For instance, the belt 10 comprises a plexiglas plaque 21, in which the three non aligned interdependent radio-opaque markers are fixed.

The three markers M₁, M₂ and M₃ belong to a same plane, therefore at least two 2D different X-ray projections 2DXR₁ and 2DXR₂ acquired with different orientation angles θ₁ and θ₂ of the C-arm system 5 are needed in order to determine the position of the ultrasound probe in the X-Ray referential (O, x, y, z). However, since the three markers are interdependent, and non-aligned, which means that they form a rigid tetraedre, it is well-known to those skilled in the art that the position of the probe is completely specified by the two different X-ray projections 2DXR₁ and 2DXR₂.

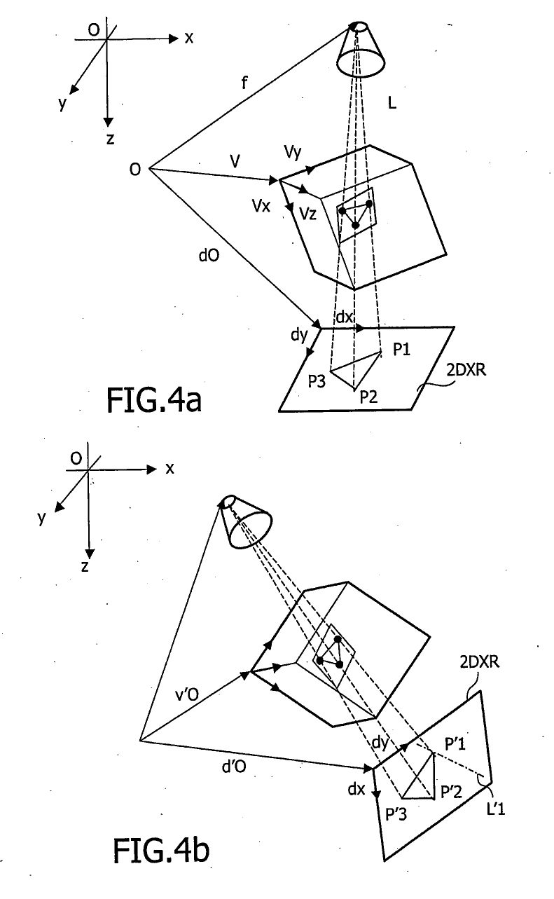

Referring to FIGS. 4a and 4b, we consider the detector referential (dO, dx, dy). It will appear to those skilled in the art that six parameters like for instance the coordinates (dx₁, dy₁), (dx₂, dy₂), (dx₃, dy₃) of the projections P₁, P₂, P₃ of the three markers M₁, M₂ and M₃ in the first 2D X-ray image 2DXR₁ and the coordinates (d′x₁, d′y₁), (d′x₂, d′y₂), (d′x₃, d′y₃) of the projections P′₁, P′₂, P′₃ of the three markers M₁, M₂ and M₃ in the second 2D X-ray image 2DXR₂ do completely specify the position of the ultrasound probe 9 in the X-Ray referential (O, x, y, z), provided that the difference of orientation angle between the two X-ray projections is known. Moreover, it should be noted that the localized points P₁, P₂, P₃ and P′₁, P′₂ and P′₃ follow epipolar constraints: this means for instance that a line L₁ linking the source focal point to the point P₁ appears as a projected line L′₁ in the second X-ray image 2DXR₂, which comprises P′₁. A first advantage is that P′₁ has not to be searched within the whole image, but only on the projected line L′₁. A second advantage is that it gives a way of associating the points P₁, P₂, P₃ and P′₁, P′₂, P′₃ with the right markers M₁, M₂ and M₃.

An advantage of the radio-opaque markers M₁, M₂ and M₃ is to appear in a 2D X-projection with a very high contrast, which makes their localization easy and precise. Such a localization may be achieved manually or automatically. In the manual case, a user may click on at least two radio-opaque markers in each 2D X-ray projections. In the automatical case, image processing techniques well known to those skilled in the art, like for instance a morphological filter, may be used for detecting the radio-opaque markers, which appear as highly contrasted blobs in the 2D X-ray projections.

It should be noted that such a localization of the ultrasound probe 9 is firstly handled in a preoperative step of a clinical procedure. As a matter of fact, with the invention, there is a priori no need to move the ultrasound probe 9 during the clinical procedure, because the large field of view of the X-ray acquisition system allows a visualization of the whole part of the patient body concerned by the clinical procedure. However, unwanted motion of the probe may occur due to a patient movement. Therefore, in order to avoid any error accumulation, the probe localization has to be repeated regularly during the clinical procedure.

Once the ultrasound probe 9 has been located in the X-Ray referential (O, x, y, z), an orientation of the probe is known and therefore, the location of the 3D ultrasound data set 22, also called 3D ultrasound cone, can be deduced. This is achieved by the converting means which calculate a position of a point of said 3D ultrasound data set in the X-Ray referential from said ultrasound probe localization. A projection of said point on the detector can also be deduced.

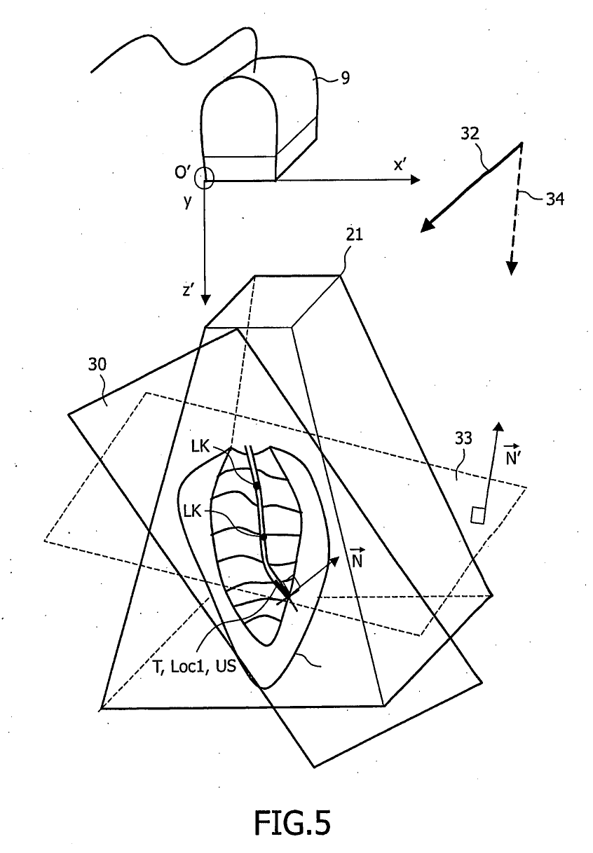

Referring to FIG. 5, the first localization means 12 are intended to provide a first localization Loc_{1,US} of the medical instrument in the 3D ultrasound data set within the referential of the ultrasound acquisition means (O′, x′, y′, z′). The detection means allow to automatically define a crop plane 30 by the detected point T and a normal orientation \vec{N}, which corresponds to the known orientation 32 of the X-ray source 6. An advantage is that, in view of generating a bimodal representation of the medical instrument, the crop plane 30 can be used for delimitating a subvolume of interest within the 3D ultrasound data set and for removing all other data, which could occlude structures of interest like the medical instrument 4. This predefined crop plane 30 may also be advantageously rotated for searching a viewing angle view within the 3D ultrasound data set from which the medical instrument is more visible. A rotated crop plane is obtained. Advantageously said viewing angle is applied to the C-arm system in order to optimize the 2D X-ray image.

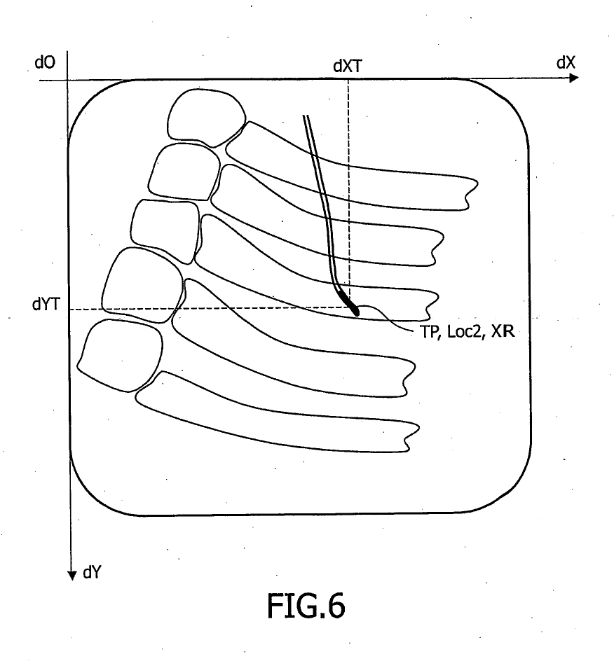

Referring to FIG. 6, the second localization means 13 are intended to provide a second localization Loc_{2,XR} of the projection of the medical instrument in the 2D X-ray image within the detector referential (dO, dx, dy) in accordance with the X-Ray geometry.

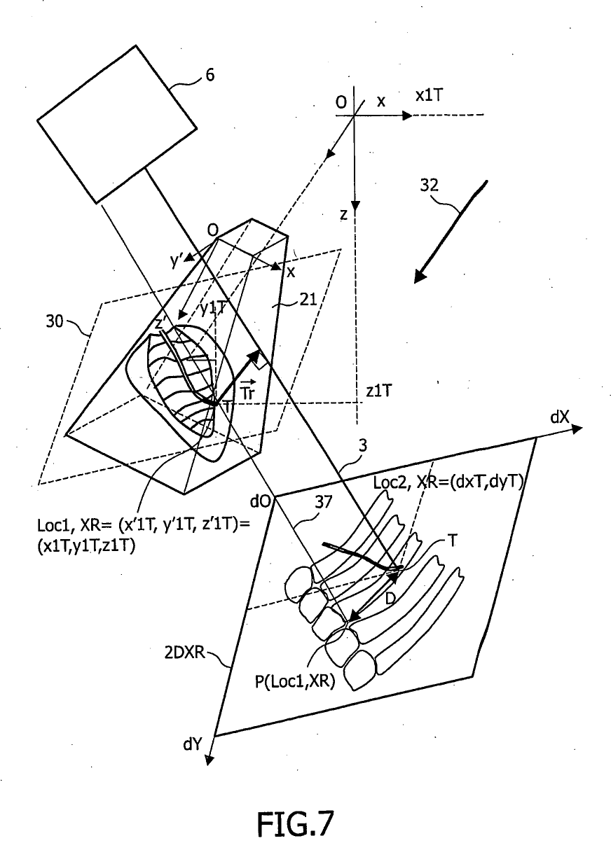

Referring to FIG. 7, the first ultrasound localization Loc_{1,US} within said referential of the ultrasound acquisition means is converted into a first X-ray localization Loc_{1,XR} within the X-Ray referential by the converting means 14.

The localizations Loc_{1,XR} and Loc_{2, XR} are further used by the mapping means 15 for defining a transformation Tr, which maps the 3D ultrasound data set with said 2D X-ray image. A mapped 3D ultrasound data set is obtained. Such a transformation is defined such that a distance between the projection of the first X-Ray localization on the 2D X-ray image in accordance with the X-Ray geometry and the second X-Ray localization is minimized.

It should be noted that the first and second X-Ray localizations Loc_{1, XR}, Loc_{2,XR} may comprise several features like a position of a landmark in the X-Ray referential, an orientation of the medical instrument or any other characteristic of the shape of the medical instrument 4. Consequently, the way in which such a distance is measured may depend on the features used for defining the first and second localizations. With a single landmark, an Euclidean distance may be sufficient. With a plurality of landmarks, functions of distance, which are well known to those skilled in the art, may advantageously be used.

It should be also noted that these first and second localizations Loc_{1, XR}, Loc_{2,XR} of the medical instrument are obtained in real-time and continuously during the clinical procedure, thus allowing a real-time mapping of the 3D ultrasound data set with the 2D X-ray image, which is based on a tracking of the medical instrument 4.

The medical instrument usually comprises a tip T at its extremity. In particular, an electrophysiology catheter comprises a metal tip, which is very echogen and leaves a specific signature in the 3D ultrasound data set. Such a metal tip is also strongly radio-opaque. Therefore, such a metal tip presents a high contrast both within the 3D ultrasound data set and the 2D X-ray image and can be advantageously considered as a valuable landmark. In addition, the tip of a catheter is a small and thin segment. Therefore, either the tip end is considered as a punctual landmark or the whole tip is considered in order to specify at least a punctual landmark and an orientation of the medical instrument.

Therefore, the detection means in accordance with the invention involve image processing techniques, which are well known to those skilled in the art, for enhancing either a highly contrasted punctual blob or a highly contrasted segment in a relatively uniform background.

In a first embodiment of the invention illustrated by FIGS. 5 and 6, the localization means 12, 13 comprise means for detecting the tip end of the medical instrument 4. In the following the tip end will be denoted T within the 3D ultrasound data set and the tip projection will be denoted T_{P} in the 2D X-ray image. The tip end T is detected at a position (x_{1T}, y_{1T}, z_{1T}) in the X-Ray referential and the projection T_{P} of the tip is detected at a position (dxT, dyT) in the detector referential (dO, dx, dy). In the first embodiment of the invention, the first and second localizations Loc_{1, XR}, Loc_{2,XR} are based on the respective positions of the unique landmark T and its projection T_{P} provided by the detection means.

Consequently, from the knowledge of these first and second localizations Loc_{1, XR}, Loc_{2,XR}, the mapping means 15 in accordance with the first embodiment of the invention are capable of defining a translation for minimizing the distance D between a projection P(Loc_{1, XR}) of the first X-Ray localization Loc_{1, XR} and the second X-Ray localization Loc_{2,XR}, as shown in FIG. 7. Such a projection P(Loc_{1, XR}), which is defined by the geometry of the X-Ray acquisition means, belongs to a projection line 37 passing through the tip end T. An advantage of this first embodiment of the invention is that it is very simple.

The translation defined by the transformation means is specified by a vector \vec{T}r, which connects the tip T to the projection line 36. It turns out that a plurality of translations can be derived from such a definition. Preferably, the chosen translation is the one which minimizes a 3D displacement of the first X-Ray localization Loc_{1, XR}. This particular translation is defined by the vector \vec{T}r, which is perpendicular to the projection line 36.

It should be noted that, due to the conic geometry of the X-Ray acquisition system, the vector \vec{T}r is not necessarily included in the crop plane 30.

In an alternative of the first embodiment of the invention, the whole tip is detected, which allows to determine a location of a landmark, for instance the tip end T and an orientation of the tip, specified by two Euler angles. Advantageously, a transformation comprising a translation and two rotations can be derived and the mapping of the 3D ultrasound data set with the 2D X-Ray image is improved.

In a second embodiment of the invention also illustrated by FIG. 5, the first and second localizations of the medical instrument 4 are based on the detection of a plurality, i.e. at least three non aligned landmarks T, Lk₂, Lk₃, which are arranged on the medical instrument 4. Such a plurality of landmarks allow to define a second crop plane 33 and a second normal \vec{N}′ within the 3D ultrasound data set, which can advantageously serve for reorienting the X-ray source 6 in order to optimize the X-ray acquisition with respect to the detected position of the medical instrument 4. An advantage of the second embodiment of the invention is that it allows to define a transformation having six degrees of freedom, i.e. a translation and three angles. Such a transformation completely specifies the displacement of the 3D ultrasound data set in the X-Ray referential. Therefore, the mapping of the 3D ultrasound data set with the 2D X-Ray image is made more precise.

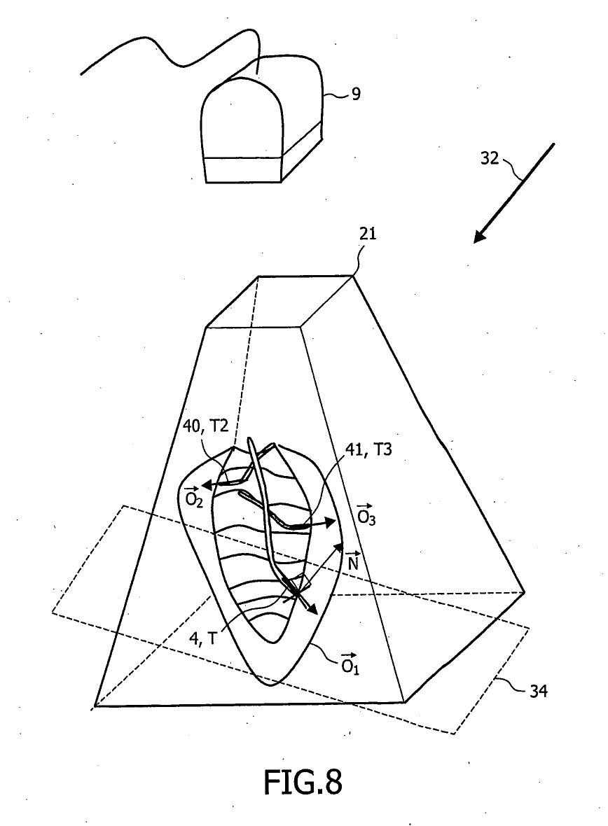

In a third embodiment of the invention illustrated by FIG. 8, the plurality of landmarks are distributed over the medical instrument 4 and at least two reference medical instruments 40, 41. Said reference medical instruments 40, 41 are both fixed in the patient body during the whole clinical procedure and comprise each an echogen and radioopaque tip T₂, T₃. They may also comprise other landmarks than the tips T, T₂, T₃, which may allow for instance the determination of tip orientations \vec{O}₁, \vec{O}₂, \vec{O}₃.

A first advantage of the third embodiment of the invention is that the landmarks used for localizing the medical instrument are more distant from each other. Therefore, the definition of the transformation is more robust to local errors of localization. As a matter of fact an error of one or two pixels has no consequences at a vicinity of the medical instrument, but can have dramatical effects in more distant areas of the 3D ultrasound data set.

A second advantage of using landmarks which are located on the reference medical instruments is that, unlike the medical instrument 4, they are fixed with respect to the anatomy. Consequently, any displacement of a landmark of a reference medical instrument with respect to the anatomy may advantageously considered as an indication that the ultrasound probe has moved and more generally that the mapping of the 3D ultrasound data set with the 2D X-ray image is no more reliable and accurate. Notably, if one of the landmarks of a reference medical instrument is no more visible within the bimodal representation BI at a time t, a consequence is that the whole processus should be repeated, i.e. a new localization of the ultrasound probe within the X-Ray referential should be performed. However, if none of the landmarks has disappeared at a time t, but was only displaced with respect to its localization at a time t0, a motion compensation of the 3D ultrasound data set between times t and t0 should be sufficient.

It should be noted that for all the previously described embodiments of the invention, the transformation is preferably chosen such that it minimizes a 3D displacement of the first X-Ray localization Loc_{1,XR}. An advantage is that such a transformation, which is intended to provide a small correction of a previous mapping of the 3D ultrasound data set and the 2D X-Ray image, ensures that the landmarks of the first X-Ray localization will still be associated with the right landmarks of the second X-Ray localization of the medical instrument.

The generation and display means 16 in accordance with the invention are intended to generate a bimodal representation BI of the medical instrument 4, in which information coming from both the 2D X-ray image 2DXR and the transformed 3D ultrasound data set are combined.

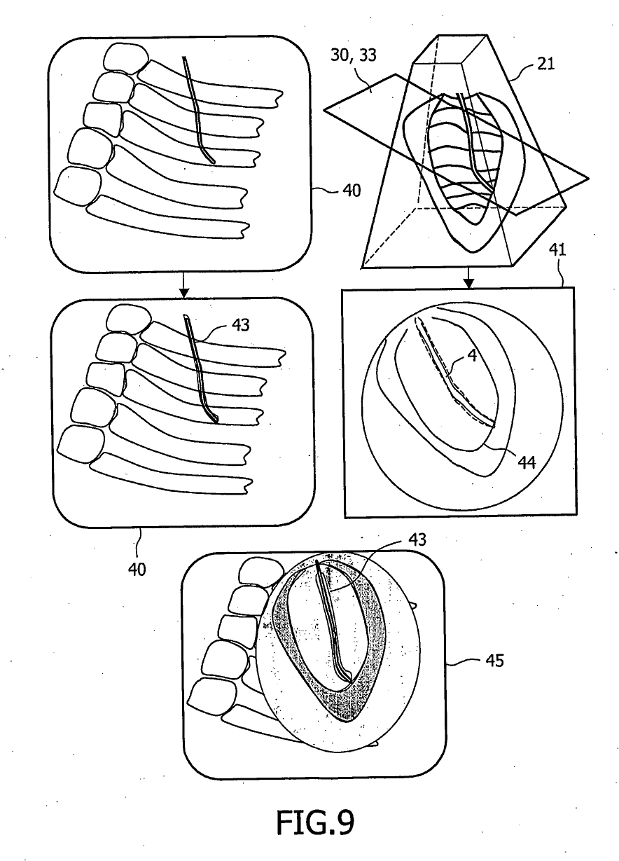

Preferably, such a combination is X-Ray driven, which means that it is made on the basis of a 2D X-Ray image 40, as shown in FIG. 9.

Advantageously a 2D ultrasound view 41 corresponding to the ultrasound information contained in one of the previously defined crop planes 30, 33 including at least part of the medical instrument 4 is extracted from the 3D ultrasound data set 21 acquired at a time t.

A correspondance between points included into the 2D ultrasound view 41 and points included into the 2D X-ray image 40 can be calculated from the knowledge of the localization of the ultrasound probe 9 within the X-Ray referential (O, x, y, z) provided by the probe localization means 11.

The bimodal projection is for instance formed such that the intensity values of all the points of the 2D X-ray projection 40 which have a corresponding point in the 2D ultrasound view 41 are replaced. An advantage is that the bimodal projection 45 obtained offers both an improved visibility of the surrounding tissues.

It is well known to those skilled in the art that the projection of the medical instrument given by the X-ray source 6 on the detector 7 is of good quality and benefits from high resolution and contrast. A position of the projection of the medical instrument 4 within the 2D X-ray projection 40, that is in the detector referential (dO, dx, dy), can be derived from the position of the medical instrument in the X-Ray referential (O, x, y, z) given by the localization of the medical instrument within the 3D ultrasound data set by the ultrasound localization means 12, the. This position is for instance a set 43 of points of the X-ray projection corresponding to a set of points 42 within the 2D ultrasound view 41.

Advantageously, the intensity values of the points of the 2D X-ray projection 40 belonging to the detected medical instrument are not replaced by the corresponding ultrasound intensity values. An advantage is to keep the good visibility and resolution of the medical instrument provided by-the X-ray acquisition means.

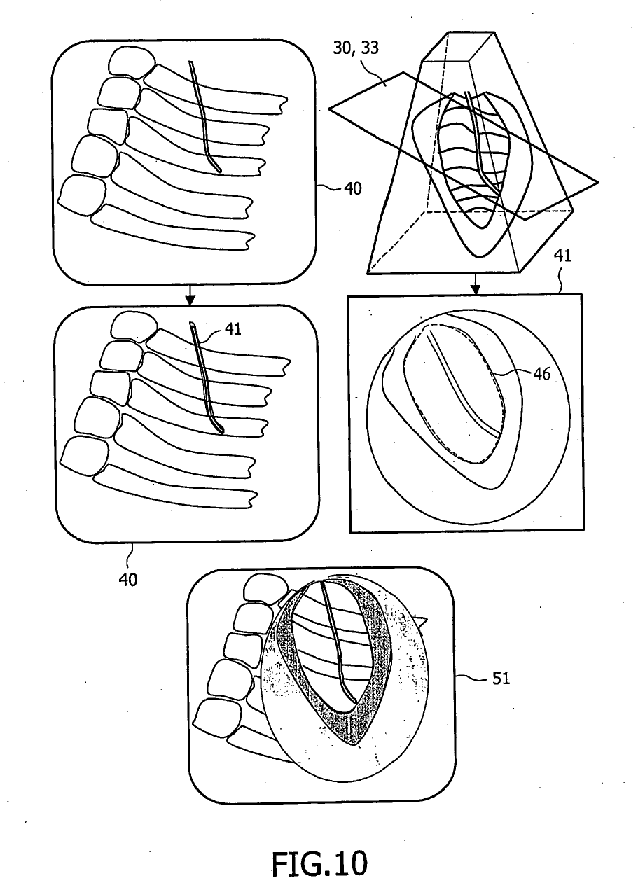

In an alternative shown in FIG. 10, the system in accordance with the invention further comprises means for segmenting a wall tissue region, for instance the endocardiac wall 44 in the neigbourhood of the medical instrument 4. This is achieved by image processing techniques, well known to those skilled in the art, such as intensity value thresholding, since wall tissues like myocardium appear brighter than blood in ultrasound images.

Another possibility is to use an active contour technique (also called “snake”). This technique, well known to those skilled in the art, firstly consists in defining an initial contour and secondly in making said initial contour evolve under the influence of internal and external forces. A final contour 46 is obtained. It is then possible to differentiate points located inside from points located outside the contour 46 and to replace only the outside points of the 2D X-ray projection 40 by the corresponding points of the 2D ultrasound view 41. An advantage of this second embodiment is to benefit from X-ray information in a larger neighbourhood of the medical instrument 4.

In another alternative of the invention, an alpha blending technique, well-known to those skilled in the art, is used for combining the X-ray intensity values of the points of the X-ray projection with the ultrasound intensity values of the corresponding points of the 3D ultrasound data set. An advantage is that this alternative is very simple to implement.

It should be noted that the generation means 16 could inversely generate a bimodal representation on the basis of the 3D ultrasound data set and replace X-ray information by ultrasound information. However, it is of less interest, because in this case, the bimodal representation has an image field which is reduced to the one of the 3D ultrasound acquisition means.

It should be noted that the system in accordance with the invention presents a particular interest for electrophysiology procedures, which consist either in generating an electrical activation map of a heart cavity wall for diagnosing heart diseases or in burning a zone of the wall tissue, which has been identified as abnormal. As a matter of fact, the system in accordance with the invention both provides a live visualisation of a large viewing field of the intervention, in which the medical instrument, the bone structures and the surrounding wall tissues are simultaneously visible and a live localization of the medical instrument, allowing a generation of the electrical activation map without further operation.

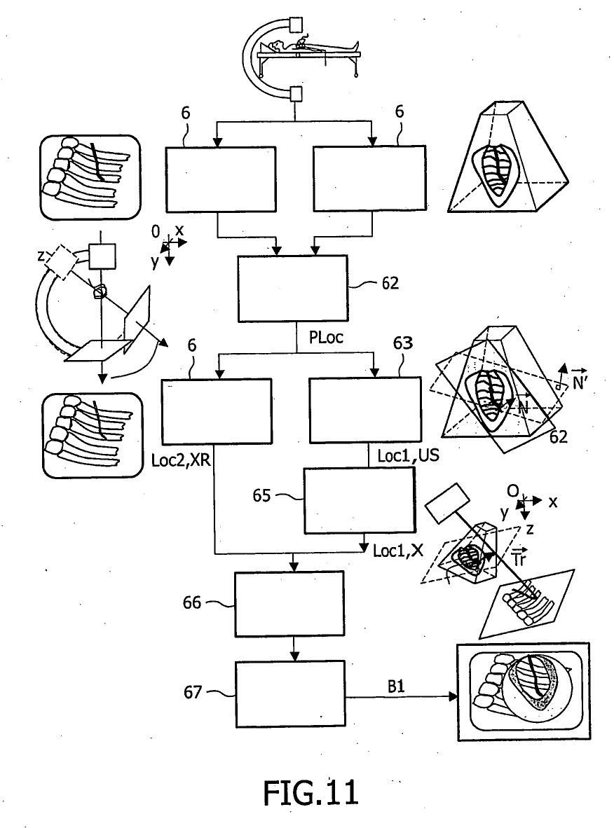

The invention also relates to a method of guiding a medical instrument 4 in a patient body. Referring to FIG. 11, such a method comprises the steps of:
acquiring 60 at least a two-dimensional X-ray image, said two-dimensional X-ray image comprising a projection of said medical instrument in accordance with a geometry of said X-ray acquisition system,
acquiring 61 a three-dimensional ultrasound data set of said medical instrument 4 using said ultrasound probe 9,
localizing 62 said ultrasound probe in a referential (O, x, y, z) of said X-ray acquisition system,
providing 63 a first localization Loc_{1, US} of said medical instrument 4 within a referential (O′,x′,y′,z′) of said 3D ultrasound acquisition means,
converting 65 said first localization Loc_{1, US} within said referential of the 3D ultrasound data set into a first converted localization Loc_{1, XR} within said referential of the X-ray acquisition system,
providing 64 a second localization Loc_{2, XR} of said projection of the medical instrument in said two-dimensional X-ray image within the referential (dO, dx, dy) of said 2D X-ray image,
mapping 66 said three-dimensional ultrasound data set with said two-dimensional X-ray image in accordance with a transformation, which minimizes a distance between a projection of said first X-Ray localization on said two-dimensional X-Ray image in accordance with said geometry of the X-Ray acquisition means and said second localization,
generating and displaying 67 a bimodal representation of said medical instrument 4 in which both 2D X-ray image and said mapped 3D ultrasound data set are combined.

The drawings and their description hereinbefore illustrate rather than limit the invention. It will be evident that there are numerous alternatives, which fall within the scope of the appended claims. In this respect the following closing remarks are made: there are numerous ways of implementing functions by means of items of hardware or software, or both. In this respect, the drawings are very diagrammatic, each representing only one possible embodiment of the invention. Thus, although a drawing shows different functions as different blocks, this by no means excludes that a single item of hardware or software carries out several functions, nor does it exclude that a single function is carried out by an assembly of items of hardware or software, or both.

Any reference sign in a claim should not be construed as limiting the claim. Use of the verb “to comprise” and its conjugations does not exclude the presence of elements or steps other than those stated in a claim. Use of the article “a” or “an” preceding an element or step does not exclude the presence of a plurality of such elements or steps.

1. A medical system comprising:
a medical instrument to be guided in a patient body,
X-Ray acquisition means for acquiring a two-dimensional X-ray image comprising a projection of said medical instrument in accordance with a geometry of said X-Ray acquisition means,
ultrasound acquisition means for acquiring a three-dimensional ultrasound data set of said medical instrument using an ultrasound probe,
means for localizing said ultrasound probe within a referential of the X-ray acquisition means,
means for providing a first ultrasound localization of said medical instrument within a referential of said ultrasound acquisition means,
means for converting said first ultrasound localization within said referential of the ultrasound acquisition means into a first X-ray localization within said referential of the X-ray acquisition means, using said localization of the ultrasound probe,
means for providing a second X-ray localization of said projection of the medical instrument in a referential of said two-dimensional X-ray image,
means for mapping said three-dimensional ultrasound data set with said two-dimensional X-ray image in accordance with a transformation, which minimizes a distance between a projection of said first X-ray localization on said two-dimensional X-Ray image in accordance with said geometry of the X-Ray acquisition means and said second X-ray localization,
means for generating and displaying a bi-modal representation of said medical instrument in which said two-dimensional X-ray image and said mapped three-dimensional ultrasound data set are combined.

2. A system as claimed in claim 1, wherein said means for providing a first ultrasound localization and said means for providing a second X-Ray localization of said medical instrument comprise detection means for detecting localization features of said medical instrument.

3. A system as claimed in claim 2, wherein said localization features comprise a landmark of said medical instrument.

4. A system as claimed in claim 3, wherein said transformation comprises a translation.

5. A system as claimed in claim 2, wherein said localization features comprise a plurality of landmarks of said medical instrument.

6. A system as claimed in claim 5, wherein said transformation comprises a translation and three rotations.

7. A system as claimed in claim 1, wherein said transformation is intended to minimize a three-dimensional displacement of said first X-Ray localization.

8. A system as claimed in claim 5, wherein said plurality of landmarks belongs to said medical instrument and to at least a first and a second reference medical instruments.

9. A system as claimed in claim 1, wherein said ultrasound probe localization allows to define a crop plane, which delimitates in the 3D ultrasound data set data to be removed from data to be used by the generating and display means for generating said bimodal representation.

10. A method of guiding a medical instrument in a patient body, comprising the steps of:
acquiring a two-dimensional X-ray image using an X-ray acquisition system, said two-dimensional X-ray image comprising a projection of said medical instrument in accordance with a geometry of said X-ray acquisition system,
acquiring a three-dimensional ultrasound data set of said medical instrument using said ultrasound probe,
localizing said ultrasound probe in a referential of said X-ray acquisition system,
providing a first localization of said medical instrument within a referential of said 3D ultrasound data set,
converting said first localization within said referential of the 3D ultrasound data set into a first X-Ray localization within said referential of the X-ray acquisition system,
providing a second localization of said projection of the medical instrument in a referential of the two-dimensional X-Ray image,
mapping said three-dimensional ultrasound data set with said two-dimensional X-ray image in accordance with a transformation, which minimizes a distance between a projection of said first X-Ray localization on said two-dimensional X-Ray image in accordance with said geometry of the X-Ray acquisition means and said second localization,
generating and displaying a bimodal representation of said medical instrument in which both 2D X-ray image and said mapped 3D ultrasound data are combined.
